(19) United States
(12) Patent Application Publication
(10) Pub. No.: US 2024/0065671 A1
(43) Pub. Date: Feb. 29, 2024

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Tsuyoshi MATSUMOTO, Tokyo (JP)

(73) Assignee: FUJIFILM Corporation, Tokyo (JP)

(21) Appl. No.: 18/506,081

(22) Filed: Nov. 9, 2023

Related U.S. Application Data
(63) Continuation of application No. PCT/JP2022/017617, filed on Apr. 12, 2022.

(30) Foreign Application Priority Data
May 18, 2021 (JP) 2021-083778

(51) Int. Cl. A61B 8/08; A61B 8/00

(52) U.S. Cl. CPC A61B 8/0891; A61B 8/0841; A61B 8/4427; A61B 8/463; A61B 8/5207

(57) ABSTRACT

In an ultrasound diagnostic apparatus and a control method of the ultrasound diagnostic apparatus of the present invention, a mode setting unit that sets association between each of a landscape posture and a portrait posture, and each of a search mode and an analysis mode. A posture sensor detects whether a posture of the apparatus main body is the landscape posture or the portrait posture, a mode switching unit switches a display mode between the search mode and the analysis mode on the basis of the posture of the apparatus main body and the association, and an information generation unit generates information for the search mode or information for the analysis mode, as information for the display mode. A display control unit displays the ultrasound image and the information for the display mode on the monitor. As a result, in a case where blood vessel puncture is performed, information necessary for blood vessel puncture is switched and displayed according to the posture of the apparatus main body.

### CROSS-REFERENCE TO RELATED APPLICATIONS

This application is a Continuation of PCT International Application No. PCT/JP2022/017617 filed on Apr. 12, 2022, which claims priority under 35 U.S.C. § 119(a) to Japanese Patent Application No. 2021-083778 filed on May 18, 2021. The above applications are hereby expressly incorporated by reference, in their entirety, into the present application.

### BACKGROUND OF THE INVENTION

### 1. Field of the Invention

The present invention relates to an ultrasound diagnostic apparatus and a control method of the ultrasound diagnostic apparatus which have a function of switching and displaying information necessary for blood vessel puncture according to a posture of an apparatus main body.

### 2. Description of the Related Art

In a case where the blood vessel puncture is performed using the ultrasound diagnostic apparatus, a technique is performed which searches an ultrasound image displayed on a monitor for a blood vessel of appropriate size and depth for puncture and placement of a catheter, carefully observes and analyzes whether or not a blood vessel can be safely punctured with the puncture needle, for example, whether or not there is a peripheral tissue such as a nerve, an artery, and an organ around the blood vessel, and whether or not there is a lesion, a blood clot, or the like, and actually performs the puncture with the puncture needle in a case where it is determined that the blood vessel can be safely punctured with the puncture needle.

Depending on a user (examiner) and a subject (patient) of the ultrasound diagnostic apparatus, the direction in which the blood vessel is searched may differ, such as a width direction or a depth direction of an ultrasound image. In a case of searching for a blood vessel in the width direction, it is convenient that the posture of the apparatus main body is a landscape posture because a wide range can be seen in the width direction, and in a case of searching for a blood vessel in the depth direction, it is convenient that the posture of the apparatus main body is a portrait posture because a wide range can be seen in the depth direction. Therefore, it is useful to be able to switch the display content of the ultrasound image according to the posture of the apparatus main body.

In a case of searching for the blood vessel in a state where the posture of the apparatus main body is the landscape posture, since the observation in the depth direction is insufficient, it is necessary to analyze whether or not blood vessel puncture can be safely performed by setting the depth of the ultrasound image deep and observing the depth direction. On the contrary, in a case of searching for the blood vessel in a state where the posture of the apparatus main body is the portrait posture, since the observation in the width direction is insufficient, it is necessary to analyze whether or not blood vessel puncture can be safely performed by setting the depth shallow and observing the width direction.

However, in order to switch the setting of the depth, it is complicated because it is necessary to operate the menu of the ultrasound diagnostic apparatus. On the other, it is more intuitive in a case where the display content of the ultrasound image can be switched according to the posture of the apparatus main body, which is desirable.

On the contrary, JP2013-165923A discloses that in an ultrasound diagnostic apparatus having a tablet-type apparatus main body, a sensor detects whether the orientation of a display surface is an upright posture or an inverted posture, an image displayed on the display surface is rotated up or down on the basis of a detection signal thereof, an examiner's screen including medical information such as comments and annotations is displayed in a case of the upright posture, and a subject' screen not including the medical information is displayed in a case of the inverted posture.

JP2011-254962A discloses an ultrasound image diagnostic apparatus that is provided with a display screen in a vertically and horizontally rotatable manner, detects the vertical and horizontal rotation of the display screen, and changes at least one of a scanning condition or a display condition.

In addition, some video sharing websites on the Internet display a video in full screen in a case where the display screen is in a landscape posture, display a video in an upper half region of the display screen and display comment information in a lower half region in a case where the display screen is in the portrait posture.

### SUMMARY OF THE INVENTION

However, JP2013-165923A and JP2011-254962A do not disclose that information necessary for blood vessel puncture is switched and displayed according to the posture of the apparatus main body in a case of performing the blood vessel puncture.

Therefore, an object of the present invention is to provide an ultrasound diagnostic apparatus and a control method of the ultrasound diagnostic apparatus which can switch and display the information necessary for the blood vessel puncture according to the posture of the apparatus main body.

In order to achieve the object, an aspect of the present invention provides an ultrasound diagnostic apparatus comprising:
an ultrasound probe; and
a handheld apparatus main body,
wherein the apparatus main body includes
   a monitor that has a display screen having a rectangular shape,
   an ultrasound image generation unit that generates an ultrasound image including a short-axis view of a blood vessel on the basis of a reception signal obtained by performing transmission and reception of an ultrasound beam with respect to a subject using the ultrasound probe,
   a mode setting unit that sets association between each of a landscape posture in which the display screen is in a laterally long state and a portrait posture in which the display screen is in a vertically long state, and each of a search mode in which information for searching for a blood vessel shown in the ultrasound image is displayed and an analysis mode in which information for performing puncture on a specific blood vessel shown in the ultrasound image is displayed,
   a posture sensor that detects whether a posture of the apparatus main body is the landscape posture or the portrait posture,
   a mode switching unit that switches a display mode between the search mode and the analysis mode on the basis of the posture of the apparatus main body and the association,
   an information generation unit that generates information for the search mode or information for the analysis mode, as information for the display mode, on the basis of the display mode, and
   a display control unit that displays the ultrasound image and the information for the display mode on the monitor.

Here, it is preferable that, in the analysis mode, in a case where the posture of the apparatus main body is the landscape posture, the display control unit displays the ultrasound image in which the specific blood vessel is shown and information regarding the specific blood vessel in a superimposed manner.

In addition, it is preferable that, in the analysis mode, in a case where the posture of the apparatus main body is the portrait posture, the display control unit displays the ultrasound image in which the specific blood vessel is shown and information regarding the specific blood vessel in a row.

In addition, it is preferable that the mode setting unit associates one of the search mode or the analysis mode with the landscape posture, and associates the other of the search mode or the analysis mode with the portrait posture.

In addition, it is preferable that the mode setting unit associates the search mode or the analysis mode with both the landscape posture and the portrait posture.

In addition, it is preferable that the mode setting unit sets the association on the basis of an instruction input from a user.

In addition, it is preferable that the mode setting unit automatically sets the association on the basis of a record of setting of the past association by a user.

In addition, it is preferable that the apparatus main body includes a blood vessel detection unit that analyzes the ultrasound image to detect a blood vessel shown in the ultrasound image.

In addition, it is preferable that the apparatus main body includes a blood vessel information acquisition unit that analyzes the ultrasound image to acquire information regarding the specific blood vessel shown in the ultrasound image.

In addition, it is preferable that the blood vessel information acquisition unit acquires at least one of a blood vessel diameter, a blood vessel depth, or a positional relationship between a blood vessel and a peripheral tissue of the specific blood vessel, as the information regarding the specific blood vessel.

In addition, it is preferable that, in the search mode, in a case where the posture of the apparatus main body is the portrait posture, the ultrasound image generation unit generates the ultrasound image with deeper depth setting than in a case where the posture of the apparatus main body is the landscape posture.

In addition, it is preferable that the information generation unit generates a first emphasis image for emphasizing each of all blood vessels shown in the ultrasound image, as the information for the search mode, and the display control unit superimposes and displays the first emphasis image corresponding to each of all the blood vessels on each of all the blood vessels.

In addition, it is preferable that the information generation unit generates a second emphasis image for emphasizing the specific blood vessel, as the information for the analysis mode, and the display control unit superimposes and displays the second emphasis image on the specific blood vessel shown in the ultrasound image.

In addition, it is preferable that the apparatus main body includes a puncture needle recognition unit that analyzes the ultrasound image to recognize a puncture needle shown in the ultrasound image, and in the analysis mode, in a case where the puncture needle is recognized, the mode setting unit switches from the analysis mode to a needle tip emphasis mode in which a needle tip of the puncture needle shown in the ultrasound image is displayed in an emphasized manner.

In addition, it is preferable that the information generation unit changes an information amount of the information for the analysis mode according to a processing capacity of the apparatus main body.

In addition, it is preferable that the apparatus main body includes a guide unit that outputs guidance for prompting a user's operation.

In addition, it is preferable that, in a case where a certain amount of time has elapsed in the search mode, the guide unit outputs guidance for prompting to change the posture of the apparatus main body.

In addition, it is preferable that, in the search mode, in a case where a motion of the ultrasound probe is smaller than a predetermined movement amount, the guide unit outputs guidance for prompting to change the posture of the apparatus main body.

In addition, it is preferable that in the search mode, in a case where the specific blood vessel enters a specific range in the ultrasound image displayed on the monitor by the user moving the ultrasound probe, the guide unit outputs guidance for prompting to change the posture of the apparatus main body.

In addition, it is preferable that, in the search mode, in a case where the user selects the specific blood vessel by a touch operation from among the blood vessels shown in the ultrasound image displayed on the monitor, the guide unit outputs guidance for prompting to move the ultrasound probe such that the specific blood vessel enters a specific range in the ultrasound image, and in a case where the specific blood vessel enters the specific range in the ultrasound image, the guide unit outputs guidance for prompting to change the posture of the apparatus main body.

In addition, it is preferable that, in a case where the posture of the apparatus main body is the landscape posture and the ultrasound probe is moved in a vertical direction by an amount greater than a predetermined movement amount, or in a case where the posture of the apparatus main body is the portrait posture and the ultrasound probe is moved in a lateral direction by an amount greater than a predetermined movement amount, the guide unit outputs guidance for prompting to change the posture of the apparatus main body.

Further, another aspect of the present invention provides a control method of an ultrasound diagnostic apparatus including an ultrasound probe, and a handheld apparatus main body having a monitor that has a display screen having a rectangular shape, the control method comprising:
a step of generating an ultrasound image including a short-axis view of a blood vessel on the basis of a reception signal obtained by performing transmission and reception of an ultrasound beam with respect to a subject using the ultrasound probe, via an ultrasound image generation unit;
a step of setting association between each of a landscape posture in which the display screen is in a laterally long state and a portrait posture in which the display screen is in a vertically long state, and each of a search mode in which information for searching for a blood vessel shown in the ultrasound image is displayed and an analysis mode in which information for performing puncture on a specific blood vessel shown in the ultrasound image is displayed, via a mode setting unit;
a step of detecting whether a posture of the apparatus main body is the landscape posture or the portrait posture, via a posture sensor;
a step of switching a display mode between the search mode and the analysis mode on the basis of the posture of the apparatus main body and the association, via a mode switching unit;
a step of generating information for the search mode or information for the analysis mode, as information for the display mode, on the basis of the display mode, via an information generation unit; and
a step of displaying the ultrasound image and the information for the display mode on the monitor, via a display control unit.

In the present invention, the posture of the apparatus main body is detected, the display mode is switched to the search mode or the analysis mode on the basis of the posture of the apparatus main body, and as the information for the display mode, the information for the search mode or the information for the analysis mode is generated and displayed on the monitor. According to the present invention, in a case where the blood vessel puncture is performed, since the information necessary for the blood vessel puncture can be obtained in each of the display modes by switching the display mode according to the posture of the apparatus main body, the user can safely and reliably perform the blood vessel puncture.

### BRIEF DESCRIPTION OF THE DRAWINGS

FIG. 1 is a block diagram of an embodiment illustrating a configuration of an ultrasound diagnostic apparatus of the present invention.

FIG. 2 is a block diagram of an embodiment illustrating a configuration of a transmission and reception circuit.

FIG. 3 is a block diagram of an embodiment illustrating a configuration of an ultrasound image generation unit.

FIG. 4 is a block diagram of an embodiment illustrating a configuration of a display mode processing unit.

FIG. 5 is a flowchart of an embodiment illustrating an operation of an ultrasound diagnostic apparatus in a case of generating an ultrasound image.

FIG. 6 is a flowchart of an embodiment illustrating an operation of an ultrasound diagnostic apparatus in a case of switching a display mode on the basis of a posture of an apparatus main body.

FIG. 7 is a conceptual diagram of an embodiment of a display screen of a monitor on which an ultrasound image is displayed in a case where a posture of an apparatus main body is a landscape posture in a search mode.

FIG. 8 is a conceptual diagram of an embodiment of a display screen of a monitor on which an ultrasound image is displayed in a case where a posture of an apparatus main body is a portrait posture in an analysis mode.

FIG. 9A is a conceptual diagram of an embodiment of a display screen of a monitor on which an ultrasound image is displayed in a case where a posture of an apparatus main body is a portrait posture and setting of a depth is 2 cm, in a search mode.

FIG. 9B is a conceptual diagram of an embodiment of a display screen of a monitor on which an ultrasound image is displayed in a case where a posture of an apparatus main body is a portrait posture and setting of a depth is 4 cm, in a search mode.

FIG. 10 is a conceptual diagram of an embodiment of a display screen of a monitor on which an ultrasound image is displayed in a case where a posture of an apparatus main body is a landscape posture in an analysis mode.

### DESCRIPTION OF THE PREFERRED EMBODIMENTS

Hereinafter, an ultrasound diagnostic apparatus and a control method of the ultrasound diagnostic apparatus according to an embodiment of the present invention will be described in detail on the basis of preferred embodiments illustrated in the accompanying drawings.

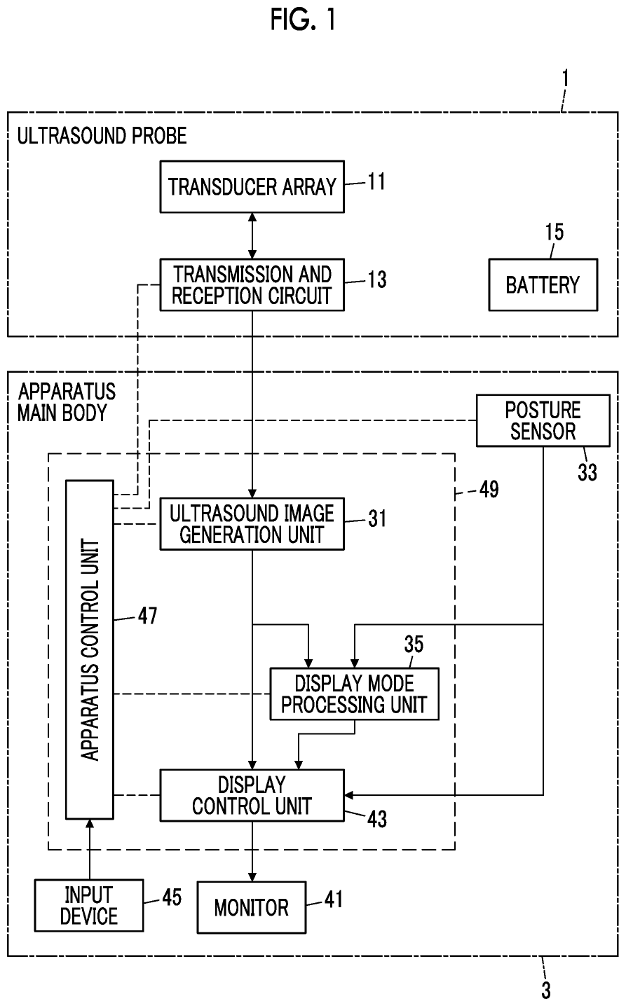

FIG. 1 is a block diagram of an embodiment illustrating a configuration of an ultrasound diagnostic apparatus of the present invention. The ultrasound diagnostic apparatus illustrated in FIG. 1 is a handheld ultrasound diagnostic apparatus, and comprises an ultrasound probe 1, and an apparatus main body 3 connected to the ultrasound probe 1. The ultrasound diagnostic apparatus of the present embodiment is realized by the ultrasound probe 1, the handheld apparatus main body 3, and an ultrasound diagnosis application program running on the apparatus main body 3.

The ultrasound probe 1 scans an examination location of a subject using an ultrasound beam, and outputs a sound ray signal corresponding to an ultrasound image of the examination location. As illustrated in FIG. 1, the ultrasound probe 1 comprises a transducer array 11, a transmission and reception circuit 13, and a battery 15. The transducer array 11 and the transmission and reception circuit 13 are bidirectionally connected to each other, and an apparatus control unit 47 of the apparatus main body 3, which will be described later, is connected to the transmission and reception circuit 13.

The transducer array 11 has a plurality of ultrasonic transducers arranged in a one-dimensional or two-dimensional manner. According to a drive signal supplied from the transmission and reception circuit 13, each of the transducers transmits an ultrasonic wave and receives a reflected wave from the subject to output an analog reception signal.

For example, each transducer is formed by using an element in which electrodes are formed at both ends of a piezoelectric body consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), or the like.

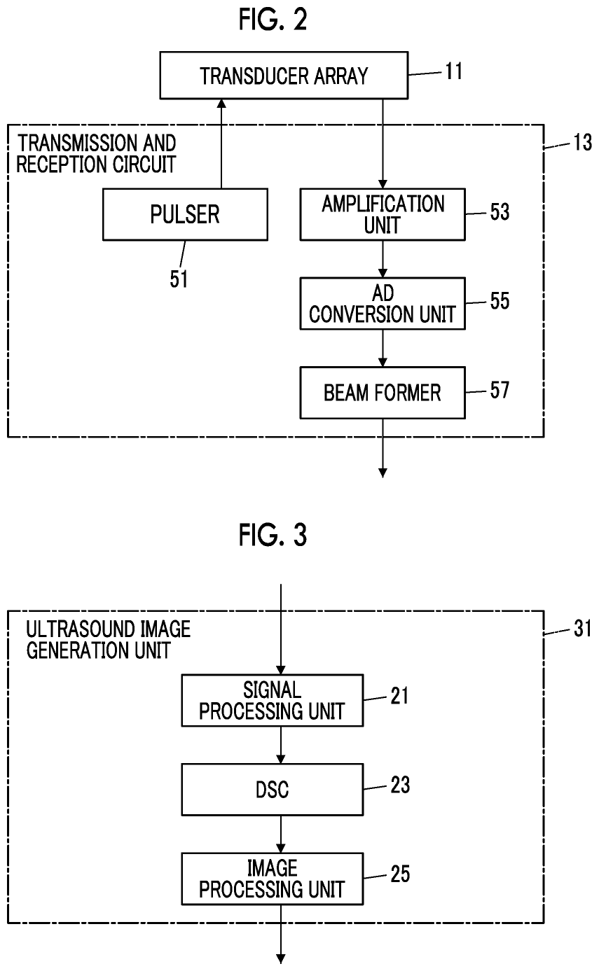

The transmission and reception circuit 13 causes the transducer array 11 to transmit the ultrasound beam, and performs reception focusing processing on the reception signal output from the transducer array 11 that has received the ultrasound echo to generate a sound ray signal, under the control of the apparatus control unit 47. As illustrated in FIG. 2, the transmission and reception circuit 13 has a pulser 51 connected to the transducer array 11, and an amplification unit 53, an analog digital (AD) conversion unit 55, and a beam former 57 that are sequentially connected in series from the transducer array 11.

The pulser 51 includes, for example, a plurality of pulse generators, and the pulser 51 performs transmission focusing processing of adjusting the amount of delay of each drive signal so that ultrasonic waves transmitted from the plurality of transducers of the transducer array 11 form an ultrasound beam on the basis of a transmission delay pattern selected by the apparatus control unit 47, and supplying the obtained signals to the plurality of transducers. By this transmission focusing processing, in a case where a pulsed or continuous-wave voltage is applied to the electrodes of the transducers of the transducer array 11, the piezoelectric body expands and contracts to generate pulsed or continuous-wave ultrasonic waves from each transducer. From the combined wave of these ultrasonic waves, an ultrasound beam is formed.

The transmitted ultrasound beam is reflected by a target, for example, a site of the subject, and propagates toward the transducer array 11 of the ultrasound probe 1. Each transducer constituting the transducer array 11 expands and contracts by receiving the ultrasound echo propagating toward the transducer array 11 in this manner, to generate the reception signal that is an electric signal, and outputs the reception signal to the amplification unit 53.

The amplification unit 53 amplifies the signals input from each transducer constituting the transducer array 11, and transmits the amplified signals to the AD conversion unit 55. The AD conversion unit 55 converts the analog signal transmitted from the amplification unit 53 into digital reception data, and outputs the reception data to the beam former 57.

The beam former 57 performs reception focusing processing in which addition is performed by giving delays to respective pieces of the reception data converted by the AD conversion unit 55 according to a sound speed distribution or a sound speed set on the basis of a reception delay pattern selected by the apparatus control unit 47. Through the reception focusing processing, a sound ray signal in which each piece of the reception data converted by the AD conversion unit 55 is phased and added and the focus of the ultrasound echo is narrowed is generated.

The battery 15 is built in the ultrasound probe 1, and supplies power to each circuit of the ultrasound probe 1.

Next, the apparatus main body 3 generates the ultrasound image of the examination location of the subject on the basis of the sound ray signal generated by the ultrasound probe 1. The apparatus main body 3 is, for example, a handheld terminal device such as a smartphone or a tablet personal computer (PC), and comprises an ultrasound image generation unit 31, a posture sensor 33, a display mode processing unit 35, a monitor 41, a display control unit 43, an input device 45, and the apparatus control unit 47 as illustrated in FIG. 1.

The ultrasound image generation unit 31 is connected to the transmission and reception circuit 13 of the ultrasound probe 1, and the display control unit 43 and the monitor 41 are sequentially connected in series to the ultrasound image generation unit 31. Each of the display mode processing unit 35 and the display control unit 43 is connected to the posture sensor 33. In addition, the display mode processing unit 35 and the display control unit 43 are sequentially connected to the ultrasound image generation unit 31. The apparatus control unit 47 is connected to the ultrasound image generation unit 31, the posture sensor 33, the display mode processing unit 35, and the display control unit 43, and the input device 45 is connected to the apparatus control unit 47.

The ultrasound probe 1 and the apparatus main body 3 are wirelessly connected by wireless communication such as Wireless Fidelity (Wi-Fi), or are connected in a wired manner by wired communication such as a Universal Serial Bus (USB) cable.

The ultrasound image generation unit 31 generates the ultrasound image (ultrasound image signal) of the examination location of the subject, from the reception signal obtained by performing transmission and reception of the ultrasound beams with respect to the examination location of the subject using the ultrasound probe 1 (more precisely, transducer array 11), in other words, from the sound ray signal generated from the reception signal by the transmission and reception circuit 13, under the control of the apparatus control unit 47. As illustrated in FIG. 3, the ultrasound image generation unit 31 has a configuration in which a signal processing unit 21, a digital scan converter (DSC) 23, and an image processing unit 25 are sequentially connected in series.

The signal processing unit 21 generates image information data corresponding to the ultrasound image on the basis of the sound ray signal generated by the transmission and reception circuit 13. More specifically, the signal processing unit 21 generates the image information data representing tomographic image information regarding tissues inside the subject, by performing envelope detection processing after signal processing, for example, correcting the attenuation of the sound ray signal generated by the beam former 57 of the transmission and reception circuit 13, which is caused by the propagation distance according to the depth of the reflection position of the ultrasonic wave.

The DSC 23 raster-converts the image information data generated by the signal processing unit 21 into an image signal according to a normal television signal scanning method.

The image processing unit 25 performs various kinds of image processing such as brightness correction, gradation correction, sharpness correction, image size correction, refresh rate correction, scanning frequency correction, and color correction according to a display format of the monitor 41, on the image signal input from the DSC 23 to generate the ultrasound image, and then outputs the ultrasound image on which the image processing has been performed, to the display control unit 43.

The posture sensor 33 detects whether the posture of the apparatus main body 3 is in a landscape posture in which the display screen of the monitor 41 is in a laterally long state or is in a portrait posture in which the display screen is in a vertically long state, under the control of the apparatus control unit 47.

The posture sensor 33 is not particularly limited as long as the posture of the apparatus main body 3 can be detected, and examples of the posture sensor 33 include an acceleration sensor that detects the motion of the apparatus main body 3, a gravity sensor that detects the gravity, and a gyro sensor that detects the rotation of the apparatus main body 3.

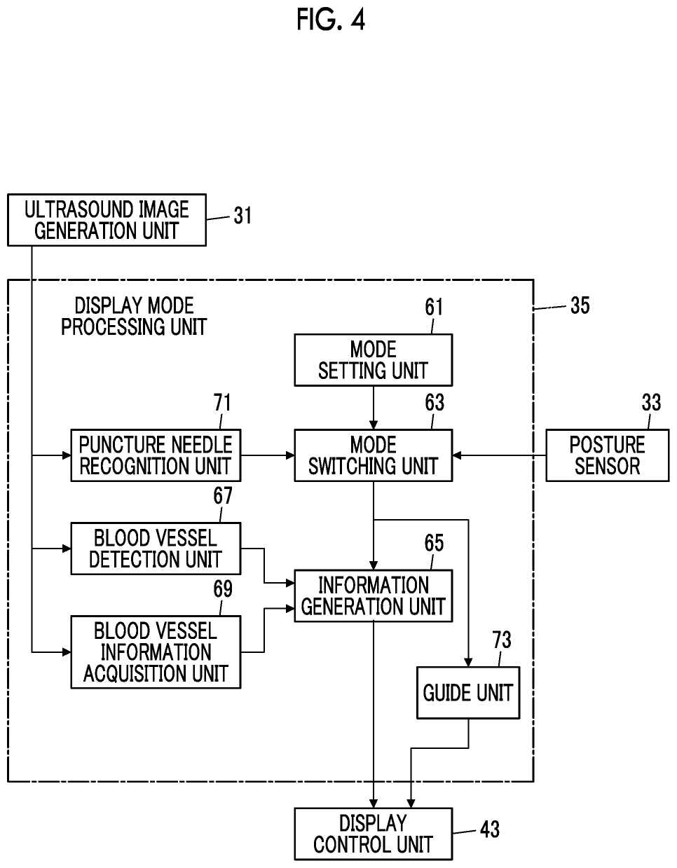

In a case where the blood vessel puncture is performed, the display mode processing unit 35 performs various kinds of processing for displaying information corresponding to the display mode, which will be described later, under the control of the apparatus control unit 47. As illustrated in FIG. 4, the display mode processing unit 35 includes a mode setting unit 61, a mode switching unit 63, an information generation unit 65, a blood vessel detection unit 67, a blood vessel information acquisition unit 69, a puncture needle recognition unit 71, and a guide unit 73.

The mode switching unit 63, the information generation unit 65, and the display control unit 43 are sequentially connected to the mode setting unit 61. The blood vessel detection unit 67, the blood vessel information acquisition unit 69, and the puncture needle recognition unit 71 are connected to the ultrasound image generation unit 31. The information generation unit 65 is connected to the blood vessel detection unit 67 and the blood vessel information acquisition unit 69. The mode switching unit 63 is connected to the puncture needle recognition unit 71 and the posture sensor 33, and the guide unit 73 and the display control unit 43 are sequentially connected to the mode switching unit 63.

The mode setting unit 61 sets the association between each of the landscape posture and the portrait posture of the apparatus main body 3, and each of a search mode and an analysis mode that are display modes.

The search mode is a display mode in which information for searching for the blood vessel shown in the ultrasound image generated by the ultrasound image generation unit 31 is displayed. The information for the search mode is not particularly limited as long as the information is necessary for the user to search for the blood vessel shown in the ultrasound image, and for example, a first emphasis image for surrounding and emphasizing regions including each of all the blood vessels shown in the ultrasound image with frame lines or emphasizing a contour of each of all the blood vessels shown in the ultrasound image can be exemplified. In addition, the information for the search mode is not limited to the display of emphasizing the blood vessel, may be any means as long as the means indicates the presence of the blood vessels, and for example, the information may be displayed as a separate map representing blood vessel positions by dots outside the display screen of the ultrasound image. In addition, as the information for the search mode, a message such as “there are xx blood vessels in the screen” or “there is a blood vessel near the center” may be read by audio, or information such as the number and position of blood vessels may be displayed as text. Furthermore, as the information for the search mode, the position of the blood vessel may be indicated by directing an arrow to the blood vessel, figures such as dots and marks may be displayed in a blood vessel region, or the region may be filled.

The analysis mode is a display mode in which information for performing puncture with the puncture needle on a specific blood vessel among the blood vessels shown in the ultrasound image is displayed. The information for the analysis mode is not particularly limited as long as the information is necessary for the user to perform the puncture on the specific blood vessel, and for example, information or the like regarding the specific blood vessel, which will be described later, can be exemplified in addition to a second emphasis image for surrounding and emphasizing a region including the specific blood vessel with the frame line or emphasizing a region in the specific blood vessel. In addition, the information for the analysis mode is not limited to the display of emphasizing the blood vessel, and may be any means as long as the means indicates the presence of the blood vessels, similar to the information for the search mode.

The mode setting unit 61 can associate a different display mode with each of the landscape posture and the portrait posture, that is, can associate one of the search mode or the analysis mode with the landscape posture, and associate the other of the search mode or the analysis mode with the portrait posture. For example, the search mode may be associated with the landscape posture and the analysis mode may be associated with the portrait posture, or the analysis mode may be associated with the landscape posture and the search mode may be associated with the portrait posture. In this case, the user can switch the display mode between the search mode and the analysis mode by changing the posture of the apparatus main body 3.

Alternatively, some users may not want to switch the display mode regardless of the posture of the apparatus main body 3. Accordingly, the mode setting unit 61 may associate the same display mode with both the landscape posture and the portrait posture, that is, associate the search mode or the analysis mode with both the landscape posture and the portrait posture. That is, the search mode may be associated with both the landscape posture and the portrait posture, or the analysis mode may be associated with the landscape posture and the portrait posture. In this case, the user can fix the display mode to the search mode or the analysis mode regardless of the posture of the apparatus main body 3.

The mode setting unit 61 can set the association between the posture of the apparatus main body 3 and the display mode on the basis of an instruction input from the user, for example.

Alternatively, the mode setting unit 61 may automatically set the association between the posture of the apparatus main body 3 and the display mode on the basis of the record (history) of the past association setting by the user.

In this case, for example, identification information for identifying the user may be assigned to each user, and the record of the past association setting by the user may be stored for each user identified by the identification information. Then, the association may be automatically set on the basis of the record of the past association setting. For example, as the past association setting, in a case where the number of times the association between the landscape posture and the search mode is set is greater than the number of times the association between the landscape posture and the analysis mode is set, the association between the landscape posture and the search mode is automatically set.

The mode switching unit 63 switches the display mode to the search mode or the analysis mode on the basis of the posture of the apparatus main body 3 detected by the posture sensor 33, and the association between the posture of the apparatus main body 3 and the display mode set by the mode setting unit 61.

For example, in a case where the landscape posture and the search mode are associated with each other and the portrait posture and the analysis mode are associated with each other, the mode switching unit 63 switches the display mode from the search mode to the analysis mode when the posture of the apparatus main body 3 is changed from the landscape posture to the portrait posture, and switches the display mode from the analysis mode to the search mode when the posture of the apparatus main body 3 is changed from the portrait posture to the landscape posture. The same applies in a case where the landscape posture and the analysis mode are associated with each other and the portrait posture and the search mode are associated with each other.

In a case where the same display mode is associated with both the landscape posture and the portrait posture, the mode switching unit 63 does not switch the display mode even in a case where the posture of the apparatus main body 3 is changed.

The information generation unit 65 generates information for the search mode or information for the analysis mode, as information for the display mode, on the basis of the display mode switched by the mode switching unit 63. That is, as the information for the display mode, the information generation unit 65 generates the information for the search mode in a case where the display mode is the search mode, and generates the information for the analysis mode in a case where the display mode is the analysis mode.

The blood vessel detection unit 67 detects the blood vessel shown in the ultrasound image by analyzing the ultrasound image. The blood vessel detection unit 67 detects short-axis views of all the blood vessels shown in the ultrasound image. The short-axis view of the blood vessel is an image of a cross section obtained by slicing the blood vessel in a cross-sectional direction perpendicular to a running direction of the blood vessel. Therefore, the short-axis view of the blood vessel represents a region (blood vessel region) of the cross section of the blood vessel in the cross-sectional direction.

The method of detecting the blood vessel is not particularly limited as long as the blood vessel shown in the ultrasound image can be detected. The blood vessel detection unit 67 can use various methods for detecting the short-axis view of the blood vessel from the ultrasound image, such as a method of using a blood vessel determination model by machine learning, and a method of using template matching, as the image analysis processing for detecting the blood vessel shown in the ultrasound image.

The blood vessel information acquisition unit 69 acquires information regarding a specific blood vessel shown in the ultrasound image by analyzing the ultrasound image.

The method of acquiring the information regarding the specific blood vessel is not particularly limited, but for example, the same method as the method of detecting the blood vessel can be used.

The information regarding the specific blood vessel is not particularly limited, but the blood vessel information acquisition unit 69 can acquire at least one of a blood vessel diameter, a blood vessel depth, or a positional relationship between the blood vessel and the peripheral tissue of the specific blood vessel, as the information regarding the specific blood vessel (vein). The peripheral tissue is also not particularly limited, but nerves, arteries, organs, and the like present in the periphery of the specific blood vessel can be exemplified.

The puncture needle recognition unit 71 recognizes the puncture needle shown in the ultrasound image by analyzing the ultrasound image. The method of recognizing the puncture needle is not particularly limited, but for example, the same method as the method of detecting the blood vessel can be used.

The guide unit 73 outputs guidance for prompting a user's operation.

The operation to prompt the user is not particularly limited, but for example, an operation of changing the posture of the apparatus main body 3, an operation of moving the ultrasound probe 1 so that the specific blood vessel as a puncture target enters a specific range in the ultrasound image displayed on the monitor 41, and the like can be exemplified.

The guidance is also not particularly limited, but may be, for example, an indicator such as a rotation mark that prompts the rotation of the apparatus main body 3, may be text data or audio data of a message that prompts the rotation of the apparatus main body 3, or may be output two or more of these at the same time. The guidance of the indicator and the text data may be displayed on the monitor 41 under the control of the display control unit 43, and the guidance of the audio data may be output as audio from a speaker (not illustrated).

The monitor (display unit) 41 has a display screen having a rectangular shape, and displays various kinds of information under the control of the display control unit 43. The monitor 41 is not particularly limited, but for example, a liquid crystal display (LCD), an organic electroluminescence (EL) display, and the like can be exemplified.

The display control unit 43 displays various kinds of information on the monitor 41 under the control of the apparatus control unit 47.

For example, the display control unit 43 displays the ultrasound image on the monitor 41 on the basis of the posture of the apparatus main body 3. That is, in a case where the posture of the apparatus main body 3 is the landscape posture, the display is performed in a portrait manner on the landscape display screen. On the other hand, in a case where the posture of the apparatus main body 3 is the portrait posture, the display is performed in the portrait manner on the portrait display screen. In this manner, even in a case where the posture of the apparatus main body 3 is the landscape posture or the portrait posture, the ultrasound image is displayed always in the portrait manner on the display screen of the monitor 41.

In addition, in a case where the posture of the apparatus main body 3 is changed from the landscape posture to the portrait posture, or in a case where the posture of the apparatus main body 3 is changed from the portrait posture to the landscape posture, the ultrasound image is rotated by 90 degrees, and is displayed always in the portrait manner on the display screen of the monitor 41.

In addition, the display control unit 43 displays the information for the display mode on the monitor 41. That is, in a case of the search mode, the information for the search mode is displayed, and in a case of the analysis mode, the information for the analysis mode is displayed.

The input device 45 receives various instructions input from the user. The input device 45 is not particularly limited, but includes, for example, various buttons, and a touch panel or the like which is provided on the display screen of the monitor 41 and through which various instructions are input by the user's touch operation.

The apparatus control unit 47 controls the ultrasound probe 1 and each unit of the apparatus main body 3 on the basis of a program stored in advance and an instruction or the like of the user input from the input device 45.

In the present embodiment, the ultrasound image generation unit 31, the display mode processing unit 35, the display control unit 43, and the apparatus control unit 47 constitute a processor 49.

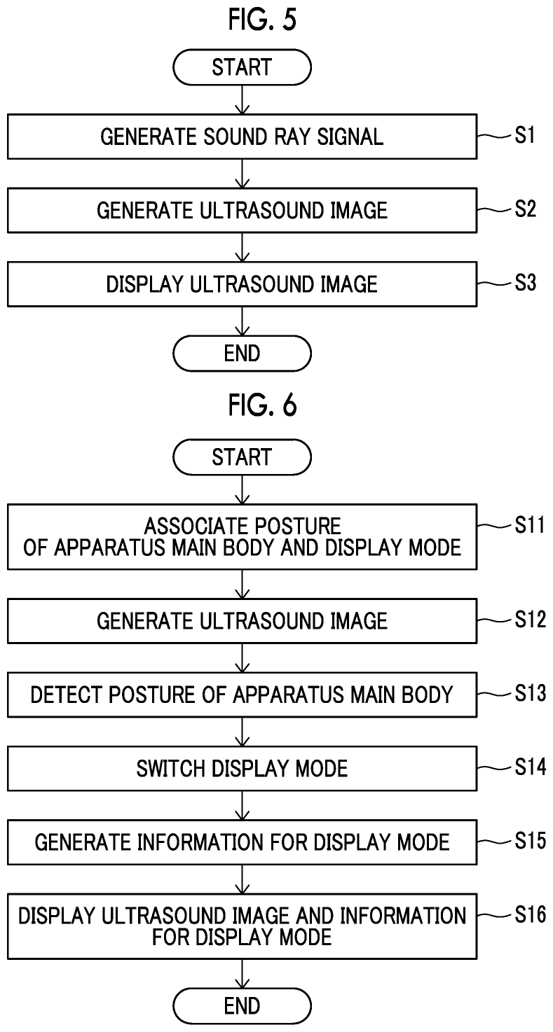

Next, the operation of the ultrasound diagnostic apparatus in a case of generating the ultrasound image will be described with reference to the flowchart of FIG. 5.

First, in a case where the ultrasound image is generated, in a state where the ultrasound probe 1 is in contact with the examination location of the subject, the transmission of the ultrasonic waves is started and the sound ray signal is generated by the transmission and reception circuit 13, under the control of the apparatus control unit 47 (Step S1).

That is, the ultrasound beams are transmitted to the examination location of the subject from a plurality of transducers of the transducer array 11 according to the drive signals from the pulser 51.

Ultrasound echoes from the examination location based on the ultrasound beams transmitted from the pulser 51 are received by each transducer of the transducer array 11, and the reception signal as an analog signal is output from each transducer of the transducer array 11, which has received the ultrasound echo.

The reception signal output from each transducer of the transducer array 11 is amplified by the amplification unit 53, and is subjected to AD conversion by the AD conversion unit 55, and thereby the reception data is acquired.

Then, by performing the reception focusing processing on the reception data by the beam former 57, the sound ray signal is generated.

Subsequently, under the control of the apparatus control unit 47, the ultrasound image of the examination location of the subject is generated by the ultrasound image generation unit 31 on the basis of the sound ray signal generated by the beam former 57 of the transmission and reception circuit 13 (Step S2).

That is, the sound ray signal generated by the beam former 57 is subjected to various kinds of signal processing by the signal processing unit 21, and the image information data representing tomographic image information regarding tissues inside the subject is generated.

Then, the image information data generated by the signal processing unit 21 is raster-converted by the DSC 23, and is further subjected to various kinds of image processing by the image processing unit 25, and thus the ultrasound image is generated.

Subsequently, under the control of the apparatus control unit 47, predetermined processing is performed on the ultrasound image generated by the image processing unit 25, by the display control unit 43, and the processed ultrasound image is displayed on the monitor 41 (Step S3).

Next, the operation of the ultrasound diagnostic apparatus in a case of switching the display mode on the basis of the posture of the apparatus main body 3 will be described with reference to the flowchart illustrated in FIG. 6.

First, the association between the posture of the apparatus main body 3 and the display mode is performed by the display mode processing unit 35, under the control of the apparatus control unit 47.

That is, for example, the association between each of the landscape posture and the portrait posture of the apparatus main body 3, and each of the search mode and the analysis mode that are display modes is set by the mode setting unit 61 on the basis of the instruction input from the user (Step S11).

The association between the posture of the apparatus main body and the display mode can be set by the mode switching unit 63, at any timing before the association is referred to.

Subsequently, the ultrasound image of the examination location of the subject is generated by the ultrasound image generation unit 31, under the control of the apparatus control unit 47. For example, in a case of performing the blood vessel puncture, the ultrasound image including the short-axis view of the blood vessel is generated by the ultrasound image generation unit 31 (Step S12).

In addition, whether the posture of the apparatus main body 3 is the landscape posture or the portrait posture is detected by the posture sensor 33, under the control of the apparatus control unit 47 (Step S13).

Subsequently, various kinds of processing for displaying the information corresponding to the display mode are performed by the display mode processing unit 35.

That is, the display mode is switched to the search mode or the analysis mode by the mode switching unit 63 on the basis of the posture of the apparatus main body 3 detected by the posture sensor 33, and the association between the posture of the apparatus main body 3 and the display mode set by the mode setting unit 61 (Step S14).

Then, the information for the search mode or the information for the analysis mode is generated as the information for the display mode by the information generation unit 65 on the basis of the display mode switched by the mode switching unit 63 (Step S15).

For example, in a case where the display mode is switched to the search mode, the ultrasound image is analyzed and all the blood vessels shown in the ultrasound image are detected by the blood vessel detection unit 67.

Then, the information for the search mode is generated by the information generation unit 65 on the basis of the blood vessel detected by the blood vessel detection unit 67.

On the other hand, in a case where the display mode is switched to the analysis mode, the ultrasound image is analyzed and the information regarding the specific blood vessel shown in the ultrasound image is acquired by the blood vessel information acquisition unit 69.

Then, the information for the analysis mode is generated by the information generation unit 65 on the basis of the information regarding the specific blood vessel acquired by the blood vessel information acquisition unit 69.

Subsequently, the ultrasound image is displayed on the monitor 41 by the display control unit 43 on the basis of the posture of the apparatus main body 3. In addition, the information for the display mode generated by the information generation unit 65 is displayed on the monitor 41 by the display control unit 43 (Step S16).

In this manner, in the ultrasound diagnostic apparatus, the posture of the apparatus main body 3 is detected, the display mode is switched to the search mode or the analysis mode on the basis of the posture of the apparatus main body 3, and as the information for the display mode, the information for the search mode or the information for the analysis mode is generated and displayed on the monitor 41. As a result, in a case where the blood vessel puncture is performed, since the information necessary for the blood vessel puncture can be obtained in each of the display modes by switching the display mode according to the posture of the apparatus main body 3, the user can safely and reliably perform the blood vessel puncture.

Next, the operation of the ultrasound diagnostic apparatus will be described with a specific example of the case of switching the display mode on the basis of the posture of the apparatus main body 3.

First, the case where the landscape posture and the search mode are associated with each other and the portrait posture and the analysis mode are associated with each other will be described.

In a case where the blood vessel puncture is performed, the user selects a blood vessel puncture mode from examination menus displayed on the monitor 41 by a touch operation, for example.

Subsequently, in a case where the user wants to comprehensively search for the blood vessels, the posture of the apparatus main body 3 is set to the landscape posture, and the blood vessel (vein) as the puncture target is searched for while moving the ultrasound probe 1 in a state where the ultrasound probe 1 is in contact with the examination location of the subject.

Accordingly, for example, the ultrasound image including the short-axis view of the blood vessel in the examination location of the subject is generated by the ultrasound image generation unit 31.

In addition, it is detected by the posture sensor 33 that the posture of the apparatus main body 3 is the landscape posture, and the display mode is switched to the search mode by the mode switching unit 63.

Subsequently, all the blood vessels shown in the ultrasound image are detected by the blood vessel detection unit 67, for example.

In the search mode, since the user searches for the blood vessel as the puncture target while moving the ultrasound probe 1 relatively large, in a case where the processing for acquiring the information regarding the specific blood vessel is performed, there is a risk that the processing cannot follow the scanning of the ultrasound probe 1 so that a delay occurs in the display of the ultrasound image. Therefore, in the case of the search mode, it is desirable not to perform the processing for acquiring the information regarding the specific blood vessel.

Subsequently, as the information for the search mode, the first emphasis image for emphasizing each of all the blood vessels detected by the blood vessel detection unit 67, for example, an image of a rectangular frame line circumscribing the vascular wall of the blood vessel of the short-axis view is generated by the information generation unit 65.

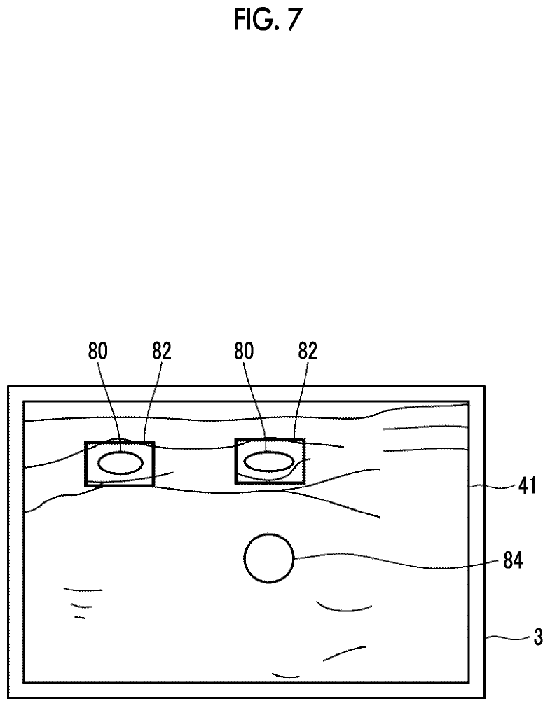

Subsequently, as illustrated in FIG. 7, in a state where the posture of the apparatus main body 3 is the landscape posture, that is, in a state where the display screen of the monitor 41 is in the laterally long state, the ultrasound image in the portrait manner is displayed on the entire screen of the display screen of the monitor 41, by the display control unit 43. In addition, in each of all blood vessels (veins) 80 shown in the ultrasound image, first emphasis images 82 respectively corresponding to all the blood vessels 80 are displayed in a superimposed manner by the display control unit 43. Furthermore, an artery 84 is displayed as an example of the peripheral tissue of the blood vessel 80.

As a result, it is possible for the user to check the blood vessel shown in the ultrasound image, and to easily decide the specific blood vessel as the puncture target.

Subsequently, in a case where the user wants to acquire the information for performing the puncture, the user moves the ultrasound probe 1 such that the specific blood vessel enters the specific range in the ultrasound image displayed on the monitor 41, for example, enters a range of a central region of the ultrasound image.

Accordingly, the ultrasound image at the position to which the ultrasound probe 1 is moved is generated by the ultrasound image generation unit 31, and is displayed on the monitor 41 by the display control unit 43.

In a case where the specific blood vessel enters the specific region in the ultrasound image displayed on the monitor 41, it is recognized that the blood vessel is the specific blood vessel as the puncture target, and the guidance for prompting to change the posture of the apparatus main body 3 is output by the guide unit 73. For example, the guidance is displayed on the monitor 41 by the display control unit 43. That is, in the search mode, in a case where the user moves the ultrasound probe 1 so that the specific blood vessel enters the specific range in the ultrasound image displayed on the monitor 41, the guide unit 73 outputs the guidance for prompting to change the posture of the apparatus main body 3.

In a case where the specific blood vessel enters the specific region in the ultrasound image, the specific blood vessel entering the specific region may be displayed by being emphasized over other blood vessels, for example, by changing the color of the frame line surrounding the region including the specific blood vessel.

Subsequently, the user changes the posture of the apparatus main body 3 from the landscape posture to the portrait posture by referring to the guidance.

In a case where the posture of the apparatus main body 3 is changed, it is detected by the posture sensor 33 that the posture of the apparatus main body 3 is the portrait posture, and the display mode is switched from the search mode to the analysis mode by the mode switching unit 63.

Subsequently, the information regarding the specific blood vessel shown in the ultrasound image is acquired by the blood vessel information acquisition unit 69.

Since the user hardly moves the ultrasound probe 1 in the analysis mode, even in a case where the processing for acquiring the information regarding the specific blood vessel is performed, the processing does not cause a delay of the display of the ultrasound image.

Subsequently, as the information for the analysis mode, a second emphasis image for emphasizing the specific blood vessel acquired by the blood vessel information acquisition unit 69, for example, an image in which a region in the specific blood vessel is colored with a predetermined color is generated by the information generation unit 65.

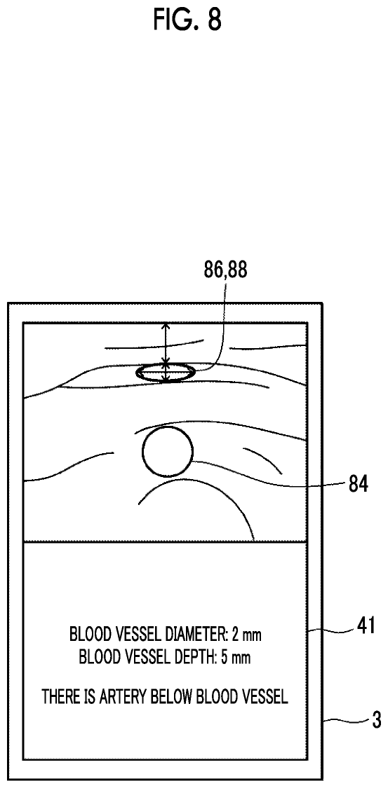

Subsequently, as illustrated in FIG. 8, in a state where the posture of the apparatus main body 3 is the portrait posture, that is, in a state where the display screen of the monitor 41 is in the vertically long state, the ultrasound image is rotated by 90 degrees, and the ultrasound image in the portrait manner is displayed on the upper half region of the display screen of the monitor 41 by the display control unit 43. In addition, as illustrated in FIG. 8, a second emphasis image 88 is displayed by being superimposed on a specific blood vessel 86 shown in the ultrasound image by the display control unit 43.

Furthermore, as illustrated in FIG. 8, as the information for the analysis mode, the information regarding the specific blood vessel 86 is displayed as text information (character information) in the lower half region of the display screen of the monitor 41, by the display control unit 43. That is, in the analysis mode, in a case where the posture of the apparatus main body 3 is the portrait posture, the ultrasound image in which the specific blood vessel 86 is shown, and the information regarding the specific blood vessel 86 are displayed in a row.

As a result, it is possible for the user to acquire the information for performing the puncture on the specific blood vessel.

In the ultrasound image illustrated in FIG. 8, arrows indicating the long diameter and the short diameter of the specific blood vessel 86, and an arrow indicating the distance from the body surface to an upper wall of the specific blood vessel 86, that is, the depth of the specific blood vessel 86 are displayed. The arrow is not actually displayed in the ultrasound image, but for example, may be displayed for the purpose of education or the like.

In addition, in the ultrasound image illustrated in FIG. 8, the artery 84 illustrated in FIG. 7 is displayed, and in order to indicate the positional relationship between the specific blood vessel (vein) 86 and the artery 84 that is the peripheral tissue thereof, the region in the artery 84 is similarly colored with a predetermined color. For example, the region in the specific blood vessel 86 is colored blue, the region in the artery 84 is colored red, and thereby the specific blood vessel 86 and the peripheral tissue thereof can be displayed in a distinguishable manner.

The method of displaying the peripheral tissue is not particularly limited, but as described above, the region in the peripheral tissue may be colored with a color different from the color for the specific blood vessel, the contour of the peripheral tissue may be displayed by being surrounded by the frame line, and the region including the peripheral tissue may be displayed by being surrounded by a rectangular frame line or the like circumscribing the peripheral tissue.

In addition, as illustrated in FIG. 8, in the lower half region of the display screen of the monitor 41, as the information regarding the specific blood vessel, “blood vessel diameter: 2 mm”, “blood vessel depth: 5 mm”, and “there is an artery below the blood vessel” are displayed. The “blood vessel diameter: 2 mm” is an average value of a long diameter and a short diameter of the specific blood vessel, but is not limited thereto, and at least one of the long diameter or the short diameter of the specific blood vessel may be displayed. In addition, the blood vessel depth is not limited to the distance from the body surface to the upper wall of the specific blood vessel, and for example, a distance from the body surface to a center position of the specific blood vessel may be displayed.

Next, the case where the portrait posture and the search mode are associated with each other and the landscape posture and the analysis mode are associated with each other will be described.

In a case where the user wants to comprehensively search for the blood vessels, the user sets the posture of the apparatus main body 3 to the portrait posture, and searches the blood vessel (vein) as the puncture target.

Accordingly, the ultrasound image including the short-axis view of the blood vessel in the examination location of the subject is generated by the ultrasound image generation unit 31.

In addition, it is detected by the posture sensor 33 that the posture of the apparatus main body 3 is the portrait posture, and the display mode is switched to the search mode by the mode switching unit 63.

Subsequently, all the blood vessels shown in the ultrasound image are detected by the blood vessel detection unit 67, and the first emphasis image is generated as the information for the search mode by the information generation unit 65.

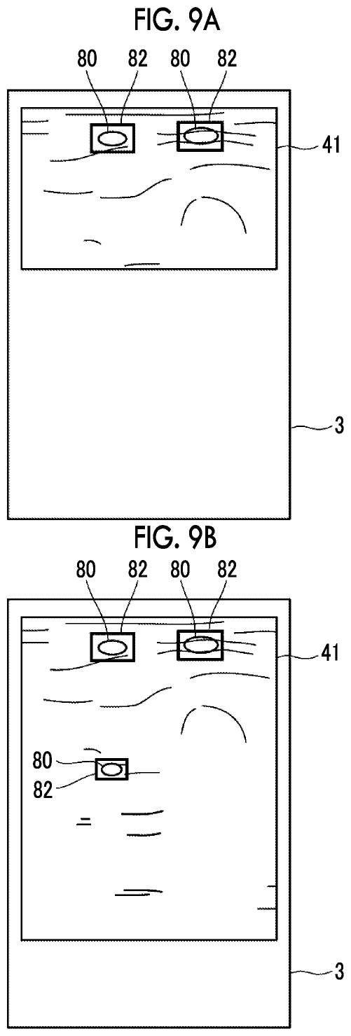

Subsequently, as illustrated in FIG. 9A, in a state where the posture of the apparatus main body 3 is the portrait posture, that is, in a state where the display screen of the monitor 41 is in the vertically long state, the ultrasound image in the portrait manner is displayed on the upper half region of the display screen of the monitor 41 by the display control unit 43. In addition, in each of all the blood vessels 80 shown in the ultrasound image, the first emphasis images 82 respectively corresponding to all the blood vessels 80 are displayed in a superimposed manner by the display control unit 43.

In the search mode, in a case where the posture of the apparatus main body 3 is the portrait posture, the size of the ultrasound image displayed on the display screen of the monitor 41 is smaller than in the case where the posture of the apparatus main body 3 is the landscape posture. However, in the search mode, since the user only needs to be able to roughly check the number of the blood vessels, and the size and depth of the blood vessel shown in the ultrasound image, the first emphasis image only needs to be displayed by being superimposed on the blood vessel shown in the ultrasound image.

In the search mode, as illustrated in FIG. 9B, in a case where the posture of the apparatus main body 3 is the portrait posture, the ultrasound image with the deeper depth setting than in a case where the posture of the apparatus main body 3 is the landscape posture may be generated by the ultrasound image generation unit 31. FIG. 9A illustrates the ultrasound image in a case where the depth setting is 2 cm as in the case where the posture of the apparatus main body 3 is the landscape posture, and FIG. 9B illustrates the ultrasound image in a case where the depth setting is 4 cm. As a result, it is possible for the user to search for the blood vessel 80 to a deeper position in the ultrasound image for which the depth setting is automatically switched without the need to operate the menu of the ultrasound diagnostic apparatus.

Subsequently, in a case where the user wants to acquire the information for performing the puncture, the user moves the ultrasound probe 1 such that the specific blood vessel enters the specific range in the ultrasound image displayed on the monitor 41.

Accordingly, the ultrasound image at the position to which the ultrasound probe 1 is moved is generated by the ultrasound image generation unit 31, and is displayed on the monitor 41 by the display control unit 43.

In a case where the specific blood vessel enters the specific region in the ultrasound image, the guidance for prompting to change the posture of the apparatus main body 3 is output by the guide unit 73, and the guidance is displayed on the monitor 41 by the display control unit 43.

Subsequently, the user changes the posture of the apparatus main body 3 from the portrait posture to the landscape posture by referring to the guidance.

In a case where the posture of the apparatus main body 3 is changed, it is detected by the posture sensor 33 that the posture of the apparatus main body 3 is the landscape posture, and the display mode is switched from the search mode to the analysis mode by the mode switching unit 63.

Subsequently, the information regarding the specific blood vessel shown in the ultrasound image is acquired by the blood vessel information acquisition unit 69, and the second emphasis image is generated as the information for the analysis mode by the information generation unit 65.

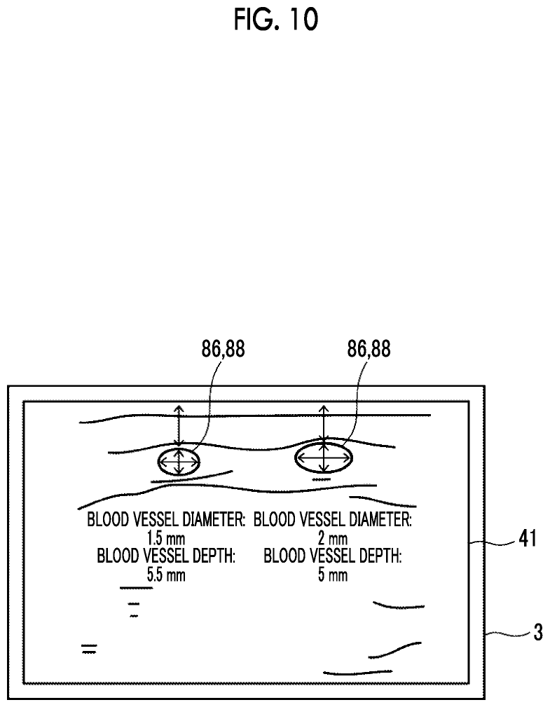

Subsequently, as illustrated in FIG. 10, in a state where the posture of the apparatus main body 3 is the landscape posture, that is, in a state where the display screen of the monitor 41 is in the laterally long state, the ultrasound image is rotated by 90 degrees, and the ultrasound image in the portrait manner is displayed on the entire screen of the display screen of the monitor 41 by the display control unit 43. In addition, as illustrated in FIG. 10, the second emphasis image 88 is displayed by being superimposed on the specific blood vessel 86 shown in the ultrasound image by the display control unit 43.

Furthermore, as illustrated in FIG. 10, as the information for the analysis mode, the information regarding the specific blood vessel 86 is displayed as text information (character information) by being superimposed on the ultrasound image by the display control unit 43. That is, in the analysis mode, in a case where the posture of the apparatus main body 3 is the landscape posture, the ultrasound image in which the specific blood vessel 86 is shown, and the information regarding the specific blood vessel 86 are displayed in a superimposed manner.

In the analysis mode, in a case where the posture of the apparatus main body 3 is the landscape posture, since the ultrasound image is displayed on the entire screen of the display screen of the monitor 41 and there is no region for displaying the information regarding the specific blood vessel, the information is displayed by being superimposed on the ultrasound image as described above. In this case, it is desirable to display the information regarding the specific blood vessel so that the information is not superimposed on the specific blood vessel such that the display of the information regarding the specific blood vessel does not interfere with the display of the specific blood vessel.

In the ultrasound image illustrated in FIG. 10, arrows similar to the arrows illustrated in FIG. 8 are displayed.

Furthermore, in the ultrasound image illustrated in FIG. 10, two veins are displayed as the specific blood vessels 86, and the information regarding the specific blood vessel 86 is displayed for each of the two veins. In a case where two or more blood vessels enter the specific range, the information regarding the specific blood vessel 86 may be displayed for each of the two or more blood vessels as described above, or since a blood vessel with a larger blood vessel diameter or a blood vessel with a shallower blood vessel depth is easier to puncture, only one blood vessel may be selected as the specific blood vessel 86 from the two or more blood vessels on the basis of at least one of the blood vessel diameter or the blood vessel depth, and the information regarding the specific blood vessel 86 may be displayed only for the one blood vessel.

In addition, as illustrated in FIG. 10, “blood vessel diameter: 2 mm” and “blood vessel depth: 5 mm” are displayed as the information regarding the specific blood vessel 86 on the right side, and “blood vessel diameter: 1.5 mm” and “blood vessel depth: 5.5 mm” are displayed as the information regarding the specific blood vessel 86 on the left side. In this example, only the blood vessel on the right side, of which the blood vessel diameter is larger and the blood vessel depth is shallower, may be selected as the specific blood vessel 86, and the information regarding the specific blood vessel 86 may be displayed only for the blood vessel on the right side.

Next, the operation of the ultrasound diagnostic apparatus in the case of performing the blood vessel puncture will be described.

As described above, in the analysis mode, in a case where the ultrasound image in which the specific blood vessel is shown and the information for the analysis mode, that is, the second emphasis image and the information regarding the specific blood vessel are displayed, the user punctures the blood vessel of the subject corresponding to the specific blood vessel shown in the ultrasound image with the puncture needle while referring to the ultrasound image and the information for the analysis mode displayed on the monitor 41.

Accordingly, by the puncture needle recognition unit 71, the ultrasound image is analyzed, and the puncture needle shown in the ultrasound image is recognized.

In a case where the puncture needle is recognized in the analysis mode, the analysis mode is automatically switched to a needle tip emphasis mode in which a needle tip of the puncture needle shown in the ultrasound image is emphasized and displayed, by the mode setting unit 61.

In a case where the blood vessel puncture is performed, since the user wants to check that the needle tip of the puncture needle has passed through the vascular wall, in the needle tip emphasis mode, it is desirable to use an emphasis image in which the vascular wall is not covered in addition to the emphasis display of the needle tip of the puncture needle in the ultrasound image.

Accordingly, in the needle tip emphasis mode, as third information for the needle tip emphasis mode, a third emphasis image which includes the specific blood vessel as in the first emphasis image in the case of the search mode and is for surrounding and emphasizing a region, which is not in contact with the vascular wall of the specific region and is larger than the specific blood vessel, with a frame line, for example, an image of a rectangular frame line surrounding the periphery of the vascular wall of the specific blood vessel without the contact with the vascular wall of the blood vessel of the short-axis view is generated by the information generation unit 65.

Then, the third emphasis image is displayed by being superimposed on the specific blood vessel shown in the ultrasound image by the display control unit 43.

As a result, it is possible for the user to check the vascular wall of the specific blood vessel, and to accurately puncture the blood vessel of the subject corresponding to the specific blood vessel with the puncture needle.

In a case where the blood vessel puncture is performed, since the user punctures the blood vessel of the subject by moving the puncture needle in real time while viewing the ultrasound image displayed on the monitor 41, no delay in the display of the ultrasound image is allowed. Therefore, in the needle tip emphasis mode, as in the case of the search mode, it is desirable not to perform the processing for acquiring the information regarding the specific blood vessel. That is, in the needle tip emphasis mode, the information regarding the specific blood vessel is not displayed.

The information generation unit 65 may change an information amount of the information for the analysis mode, for example, an information amount of the information regarding the specific blood vessel and a display amount of the information, according to the processing capacity of the apparatus main body 3.

In this case, the information generation unit 65 acquires various kinds of information including the model number, the screen size, hardware information, and the like of the apparatus main body 3 as the information on the apparatus main body 3, and estimates the processing capacity of the apparatus main body 3 on the basis of the hardware information, for example. Then, in a case where the processing capacity of the apparatus main body 3 is high, the information generation unit 65 increases the information amount according to the processing capacity of the apparatus main body 3, compared to a case where the processing capacity is low.

As a result, in various apparatus main bodies with different processing capacities, an application program that implements the present invention can be installed and used.

The guide unit 73 can output the guidance for prompting the user's operation at any timing without being limited to the case where the specific blood vessel enters the specific region in the ultrasound image displayed on the monitor 41.

For example, the guide unit 73 may output the guidance for prompting to change the posture of the apparatus main body 3 in a case where a certain amount of time has elapsed in the search mode.

As a result, the guidance prompts the user to decide a specific blood vessel, and allows the transition from the search mode to the analysis mode.

In the search mode, in a case where a motion of the ultrasound probe 1 is smaller than a predetermined movement amount, the guide unit 73 may output the guidance for prompting to change the posture of the apparatus main body 3.

In this case, it is considered that the user has decided a candidate for the specific blood vessel as the puncture target, it is possible to prompt the user to finally decide the specific blood vessel as the puncture target by outputting the guidance for prompting to change the posture of the apparatus main body 3.

The method of detecting the motion of the ultrasound probe 1 is not particularly limited, but, for example, the motion of the ultrasound probe 1 may be detected by analyzing a plurality of frames of ultrasound images, or a motion sensor, for example, an acceleration sensor may be provided to the ultrasound probe 1 and the motion of the ultrasound probe 1 may be detected by the motion sensor.

In the search mode, the user may select the specific blood vessel as the puncture target from among the blood vessels shown in the ultrasound image displayed on the monitor 41, by the touch operation or line of sight.

The method of detecting the line of sight is not particularly limited, but various line-of-sight detection techniques can be used, and for example, the line of sight can be detected from an image in which the user's eyes are captured by a camera.

In this case, first, the guide unit 73 may output the guidance for prompting to move the ultrasound probe 1 so that the specific blood vessel enters the specific range in the ultrasound image, and then, output the guidance for prompting to change the posture of the apparatus main body 3 in a case where the specific blood vessel enters the specific range in the ultrasound image.

As a result, for example, even in a case where the specific blood vessel is present at the end portion of the ultrasound image, it is possible for the user to change the posture of the apparatus main body 3 after moving the ultrasound probe 1 such that the specific blood vessel enters the specific range in the ultrasound image.

In addition, in a case where the posture of the apparatus main body 3 is the landscape posture and the ultrasound probe 1 is moved in the vertical direction by an amount greater than the predetermined movement amount, or in a case where the posture of the apparatus main body 3 is the portrait posture and the ultrasound probe 1 is moved in the lateral direction by an amount greater than the predetermined movement amount, it is considered that the user wants to search for the blood vessel as the puncture target in a direction in which the ultrasound probe 1 is moved largely. Therefore, in this case, the guide unit 73 may output the guidance for prompting to change the posture of the apparatus main body 3.

As a result, it is possible for the user to search for the blood vessel in a wider range in a direction in which the ultrasound probe 1 is moved, that is, in a direction in which the user wants to search for the blood vessel.

The apparatus main body 3 may comprise the ultrasound image generation unit 31, but the present invention is not limited thereto, and the entire ultrasound image generation unit 31 or only the signal processing unit 21 may be provided on the ultrasound probe 1 side.

In the apparatus of the embodiment of the present invention, the hardware configurations of the processing units executing various kinds of processing such as the transmission and reception circuit 13, the ultrasound image generation unit 31, the display mode processing unit 35, the display control unit 43, and the apparatus control unit 47 may be dedicated hardware, or may be various processors or computers that execute programs.

The various processors include a central processing unit (CPU) as a general-purpose processor executing software (program) and functioning as various processing units, a programmable logic device (PLD) as a processor of which the circuit configuration can be changed after manufacturing such as a field programmable gate array (FPGA), and a dedicated electric circuit as a processor having a circuit configuration designed exclusively for executing specific processing such as an application specific integrated circuit (ASIC).

One processing unit may be configured by one of the various processors or may be configured by a combination of the same or different kinds of two or more processors, for example, a combination of a plurality of FPGAs or a combination of an FPGA and a CPU). Further, a plurality of processing units may be configured by one of various processors, or two or more of a plurality of processing units may be collectively configured by using one processor.

For example, there is a form where one processor is configured by a combination of one or more CPUs and software as typified by a computer, such as a server and a client, and this processor functions as a plurality of processing units. Further, there is a form where a processor realizing the functions of the entire system including a plurality of processing units by one integrated circuit (IC) chip as typified by a system on chip (SoC) or the like is used.

Furthermore, the hardware configurations of these various processors are more specifically electric circuitry where circuit elements, such as semiconductor elements, are combined.

The method of the embodiment of the present invention can be carried out, for example, by a program for causing a computer to execute each step of the method. Further, a computer-readable recording medium in which this program is recorded can also be provided.

The present invention has been described in detail, but the present invention is not limited to the above-described embodiments, and various improvements and changes may be made within a range not departing from the scope of the present invention.

### EXPLANATION OF REFERENCES

1: ultrasound probe
3: apparatus main body
11: transducer array
13: transmission and reception circuit
15: battery
21: signal processing unit
23: DSC
25: image processing unit
31: ultrasound image generation unit
33: posture sensor
35: display mode processing unit
41: monitor
43: display control unit
45: input device
47: apparatus control unit
49: processor
51: pulser
53: amplification unit
55: AD conversion unit
57: beam former
61: mode setting unit
63: mode switching unit
65: information generation unit
67: blood vessel detection unit
69: blood vessel information acquisition unit
71: puncture needle recognition unit
73: guide unit
80: blood vessel (vein)
82: first emphasis image
84: artery
86: specific blood vessel
88: second emphasis image

What is claimed is:

1. An ultrasound diagnostic apparatus comprising:
an ultrasound probe; and
a handheld apparatus main body,
wherein the apparatus main body includes
a monitor that has a display screen having a rectangular shape,
an ultrasound image generation unit that generates an ultrasound image including a short-axis view of a blood vessel on the basis of a reception signal obtained by performing transmission and reception of an ultrasound beam with respect to a subject using the ultrasound probe,
a mode setting unit that sets association between each of a landscape posture in which the display screen is in a laterally long state and a portrait posture in which the display screen is in a vertically long state, and each of a search mode in which information for searching for a blood vessel shown in the ultrasound image is displayed and an analysis mode in which information for performing puncture on a specific blood vessel shown in the ultrasound image is displayed,
a posture sensor that detects whether a posture of the apparatus main body is the landscape posture or the portrait posture,
a mode switching unit that switches a display mode between the search mode and the analysis mode on the basis of the posture of the apparatus main body and the association,
an information generation unit that generates information for the search mode or information for the analysis mode, as information for the display mode, on the basis of the display mode, and
a display control unit that displays the ultrasound image and the information for the display mode on the monitor.

2. The ultrasound diagnostic apparatus according to claim 1,
wherein in the analysis mode, in a case where the posture of the apparatus main body is the landscape posture, the display control unit displays the ultrasound image in which the specific blood vessel is shown and information regarding the specific blood vessel in a superimposed manner.

3. The ultrasound diagnostic apparatus according to claim 1,
wherein in the analysis mode, in a case where the posture of the apparatus main body is the portrait posture, the display control unit displays the ultrasound image in which the specific blood vessel is shown and information regarding the specific blood vessel in a row.

4. The ultrasound diagnostic apparatus according to claim 1,
wherein the mode setting unit associates one of the search mode or the analysis mode with the landscape posture, and associates the other of the search mode or the analysis mode with the portrait posture.

5. The ultrasound diagnostic apparatus according to claim 1,
wherein the mode setting unit associates the search mode or the analysis mode with both the landscape posture and the portrait posture.

6. The ultrasound diagnostic apparatus according to claim 1,
wherein the mode setting unit sets the association on the basis of an instruction input from a user.

7. The ultrasound diagnostic apparatus according to claim 1,
wherein the mode setting unit automatically sets the association on the basis of a record of setting of a past association by a user.

8. The ultrasound diagnostic apparatus according to claim 1,
wherein the apparatus main body further includes a blood vessel detection unit that analyzes the ultrasound image to detect a blood vessel shown in the ultrasound image.

9. The ultrasound diagnostic apparatus according to claim 1,
wherein the apparatus main body further includes a blood vessel information acquisition unit that analyzes the ultrasound image to acquire information regarding the specific blood vessel shown in the ultrasound image.

10. The ultrasound diagnostic apparatus according to claim 9,
wherein the blood vessel information acquisition unit acquires at least one of a blood vessel diameter, a blood vessel depth, or a positional relationship between a blood vessel and a peripheral tissue of the specific blood vessel, as the information regarding the specific blood vessel.

11. The ultrasound diagnostic apparatus according to claim 1,
wherein in the search mode, in a case where the posture of the apparatus main body is the portrait posture, the ultrasound image generation unit generates the ultrasound image with deeper depth setting than in a case where the posture of the apparatus main body is the landscape posture.

12. The ultrasound diagnostic apparatus according to claim 1,
wherein the information generation unit generates a first emphasis image for emphasizing each of all blood vessels shown in the ultrasound image, as the information for the search mode, and
the display control unit superimposes and displays the first emphasis image corresponding to each of all the blood vessels on each of all the blood vessels.

13. The ultrasound diagnostic apparatus according to claim 1,
wherein the information generation unit generates a second emphasis image for emphasizing the specific blood vessel, as the information for the analysis mode, and
the display control unit superimposes and displays the second emphasis image on the specific blood vessel shown in the ultrasound image.

14. The ultrasound diagnostic apparatus according to claim 1,
wherein the apparatus main body further includes a puncture needle recognition unit that analyzes the ultrasound image to recognize a puncture needle shown in the ultrasound image, and
in the analysis mode, in a case where the puncture needle is recognized, the mode setting unit switches from the analysis mode to a needle tip emphasis mode in which a needle tip of the puncture needle shown in the ultrasound image is displayed in an emphasized manner.

15. The ultrasound diagnostic apparatus according to claim 1,
wherein the information generation unit changes an information amount of the information for the analysis mode according to a processing capacity of the apparatus main body.

16. The ultrasound diagnostic apparatus according to claim 1,
wherein the apparatus main body further includes a guide unit that outputs guidance for prompting a user's operation.

17. The ultrasound diagnostic apparatus according to claim 16,
wherein in a case where a certain amount of time has elapsed in the search mode, the guide unit outputs guidance for prompting to change the posture of the apparatus main body.

18. The ultrasound diagnostic apparatus according to claim 16,
wherein in the search mode, in a case where a motion of the ultrasound probe is smaller than a predetermined movement amount, the guide unit outputs guidance for prompting to change the posture of the apparatus main body.

19. The ultrasound diagnostic apparatus according to claim 16,
wherein in the search mode, in a case where the specific blood vessel enters a specific range in the ultrasound image displayed on the monitor by the user moving the ultrasound probe, the guide unit outputs guidance for prompting to change the posture of the apparatus main body.

20. The ultrasound diagnostic apparatus according to claim 16,
wherein in the search mode, in a case where the user selects the specific blood vessel by a touch operation from among the blood vessels shown in the ultrasound image displayed on the monitor, the guide unit outputs guidance for prompting to move the ultrasound probe such that the specific blood vessel enters a specific range in the ultrasound image, and in a case where the specific blood vessel enters the specific range in the ultrasound image, the guide unit outputs guidance for prompting to change the posture of the apparatus main body.

21. The ultrasound diagnostic apparatus according to claim 16,
wherein in a case where the posture of the apparatus main body is the landscape posture and the ultrasound probe is moved in a vertical direction by an amount greater than a predetermined movement amount, or in a case where the posture of the apparatus main body is the portrait posture and the ultrasound probe is moved in a lateral direction by an amount greater than a predetermined movement amount, the guide unit outputs guidance for prompting to change the posture of the apparatus main body.

22. A control method of an ultrasound diagnostic apparatus including an ultrasound probe, and a handheld apparatus main body having a monitor that has a display screen having a rectangular shape, the control method comprising:
a step of generating an ultrasound image including a short-axis view of a blood vessel on the basis of a reception signal obtained by performing transmission and reception of an ultrasound beam with respect to a subject using the ultrasound probe, via an ultrasound image generation unit;
a step of setting association between each of a landscape posture in which the display screen is in a laterally long state and a portrait posture in which the display screen is in a vertically long state, and each of a search mode in which information for searching for a blood vessel shown in the ultrasound image is displayed and an analysis mode in which information for performing puncture on a specific blood vessel shown in the ultrasound image is displayed, via a mode setting unit;
a step of detecting whether a posture of the apparatus main body is the landscape posture or the portrait posture, via a posture sensor;
a step of switching a display mode between the search mode and the analysis mode on the basis of the posture of the apparatus main body and the association, via a mode switching unit;
a step of generating information for the search mode or information for the analysis mode, as information for the display mode, on the basis of the display mode, via an information generation unit; and
a step of displaying the ultrasound image and the information for the display mode on the monitor, via a display control unit.
